(19) United States
(12) Patent Application Publication
(10) Pub. No.: US 2015/0066000 A1
(43) Pub. Date: Mar. 5, 2015

(54) Surgical Tool With Integrated Sensor

(71) Applicant: BRITESEED LLC, Chicago, IL (US)

(72) Inventors: Andrew An, Evanston, IL (US); Muneeb Bokhari, Chicago, IL (US); Paul Fehrenbacher, Chicago, IL (US); Jonathan Gunn, Chicago, IL (US); Mayank Vijayvergia, Chicago, IL (US)

(21) Appl. No.: 14/383,173

(22) PCT Filed: Mar. 6, 2013
(86) PCT No.: PCT/US2013/029412

§ 371 Date: Sep. 5, 2014

Related U.S. Application Data
(60) Provisional application No. 61/607,335, filed on Mar. 6, 2012.

(51) Int. Cl. A61B 17/29

(52) U.S. Cl. CPC A61B 17/29; A61B 2017/00057
USPC 606/1

(57) ABSTRACT

An instrumented surgical tool and associated systems and methods for performing surgical procedures such as tissue dissection or ligation using the instrumented surgical tool are described. In particular, a surgical tool operatively connected to a sensor used to detect a structural artifact such as the presence and characteristics of a blood vessel and to evaluate the safe use of the surgical tool within a surgical field is described.

### CROSS-REFERENCE TO RELATED APPLICATION

This application claims the benefit of U.S. Provisional Patent Application Ser. No. 61/607,335, filed Mar. 6, 2012 and entitled “Apparatus for use of blood flow to evaluate risk of tissue dissection device”, the entire disclosure of which is hereby incorporated herein by reference.

### FIELD OF INVENTION

This invention relates generally to surgical tools, systems, and methods for performing surgical procedures such as tissue dissection or ligation. In particular, this invention relates to a surgical tool operatively connected to a sensor to detect a structural artifact such as the presence and characteristics of a blood vessel and to evaluate the safe use of the surgical tool within a surgical field.

### BACKGROUND

Minimally invasive and open surgeries make use of various surgical tools to implement a variety of surgical procedures such as dissection by blade, dissection with sutures or staples to seal tissue, and energy-based tissue sealing and ablation. These surgical tools may also dissect a variety of tissues, including blood vessels. One limitation of existing vessel dissecting tools is that the user of the tool cannot always see the vessel being dissected and/or ligated. If a surgical procedure is performed on a vessel larger than allowed by the specification of the surgical tool, the vessel may not completely seal and unintended bleeding may occur as a result.

Existing sensor devices are available for use in minimally invasive and open surgical applications to identify structural artifacts and/or to analyze blood flow within the surgical field. These existing sensor devices make use of a variety of technologies including Doppler and infrared absorption to sense relevant features of the structural artifacts and/or blood flow. Although these existing sensor devices are effective at identifying vascular structures and/or other structural features, these devices are typically used separately from the surgical tools and do not communicate with the surgical tools. Further, these sensor devices are extremely difficult to use simultaneously with the surgical tools in endoscopic surgical procedures such as laparoscopy due to the limited space available within the surgical field.

This lack of visualization capability of vasculature and other structural artifacts concurrent with the use of a surgical instrument increases the risk of adverse events such as intraoperative bleeding. Improvement in blood flow analysis at regions targeted for dissection would lower the incidents of these adverse events.

Therefore, there is a need for an integrated surgical instrument and structural artifact/blood flow sensor to enhance the safety of tissue dissecting, vessel ligation, and other surgical procedures.

### SUMMARY OF THE INVENTION

In one aspect, an instrumented surgical device is provided that includes a surgical tool to perform a surgical procedure within a surgical field. The instrumented surgical device also includes a sensor operatively connected to the surgical tool. The sensor monitors the surgical field for a structural artifact.

In another aspect, a system for performing a surgical procedure on a tissue situated within a surgical field of a patient is provided. The system includes an instrumented surgical device. The instrumented surgical device includes a surgical tool to perform the surgical procedure. The surgical tool includes a functional element operatively connected to a controller. In addition, the surgical tool includes a sensor to continuously monitor the tissue within the surgical field. The sensor is operatively connected to the surgical tool.

In this other aspect, the system also includes a data post-processing module to process one or more outputs received from the sensor to generate an amount of processed data defining one or more characteristics of the tissue. The system also includes a structural artifact detection module to analyze the amount of processed data to determine an amount of artifact data characterizing one or more structural artifacts within the tissue. In addition, the system also includes an alarm signal module to assess the amount of artifact data and to generate an alarm signal if the amount of artifact data exceeds a predetermined threshold condition. Also included in the system is an alarm indication module to generate an alarm indication in response to the amount of one or more structural artifacts. Further, the system includes a GUI module to generate one or more forms. These one or more forms receive one or more inputs to the system and communicate one or more outputs from the system.

In an additional aspect, a system for performing a surgical procedure on a tissue situated within a surgical field of a patient is provided that includes an instrumented surgical device. The instrumented surgical device includes a surgical tool to perform the surgical procedure that includes a functional element operatively connected to a controller. The instrumented surgical device further includes a sensor operatively connected to the surgical tool that continuously monitors the tissue within the surgical field.

This additional aspect also includes a computing device that includes one or more processors and a CRM encoded with a surgical device application. The surgical device application includes one or more modules executable on the one or more processors.

In this aspect, the modules of the surgical device application may include: a data post-processing module to process one or more outputs received from the sensor to generate an amount of processed data defining one or more characteristics of the tissue; a structural artifact detection module to analyze the amount of processed data to determine an amount of artifact data characterizing one or more structural artifacts within the tissue; an alarm signal module to assess the amount of artifact data and to generate an alarm signal if the amount of artifact data exceeds a predetermined threshold condition; an alarm indication module to generate an alarm indication in response to the amount of one or more structural artifacts; and a GUI module to generate one or more forms. These one or more forms receive one or more inputs to the system and communicate one or more outputs from the system.

A method of performing a surgical procedure on a tissue within a surgical field is provided in another aspect. The method includes approaching the tissue with an instrumented surgical device that includes a sensor operatively attached to a surgical tool and sensing a structural artifact within the tissue using the sensor. The method further includes sending an alarm signal from the sensor to an indicator if the structural artifact exceeds a predetermined threshold condition and generating an alarm indication in response to the alarm signal using the indicator.

A laparoscopic surgical sensor is provided in yet another aspect that includes a first jaw attached to a second jaw in a hinged mechanical engagement. The first jaw includes an optical transmitter and the second jaw includes an optical receiver.

Other aspects of the invention are described in detail below.

### BRIEF DESCRIPTION OF THE DRAWINGS

FIG. 1 is an illustration of a surgical tool with a controller and functional element.

FIG. 2 is an illustration of the grasping jaws.

FIG. 3 is an illustration of the grasping jaws with integrated electrodes.

FIG. 4 is an illustration of a surgical tool with an integrated optical sensor.

FIG. 5 is a cross-sectional illustration of a surgical tool with an integrated optical sensor.

FIG. 6 is an illustration of a surgical tool with an integrated ultrasound Doppler probe.

FIG. 7 is an illustration of a surgical tool including surgical scissors with an integrated ultrasound Doppler probe.

FIG. 8 is an illustration of a surgical tool including a surgical hook with an integrated ultrasound Doppler probe.

FIG. 9 is a block diagram illustrating the elements and modules of a surgical system in one aspect.

FIG. 10 is a block diagram illustrating the elements and modules of a surgical system in a second aspect.

FIG. 11 is a flow chart illustrating a method of performing a surgical procedure using an instrumented surgical device.

FIG. 12 is a schematic diagram illustrating the elements of a surgical system.

Corresponding reference characters and labels indicate corresponding elements among the views of the drawings. The headings used in the figures should not be interpreted to limit the scope of the claims.

### DETAILED DESCRIPTION

Provided herein are surgical sensors, instrumented surgical devices, systems, and methods for monitoring a structural artifact within a surgical field concurrent with the use of a surgical tool. The instrumented surgical device may include the surgical tool to perform a surgical procedure within a surgical field and a sensor operatively connected to the surgical tool to monitor the surgical field for a surgical artifact. “Surgical field”, as defined herein, refers to an afflicted area of a patient that is treated using a surgical procedure performed by the surgical device. The structural artifact may include, but is not limited to, blood flow, tissue type, material type, or any other tissue property that may be detected by the sensor.

These instrumented surgical devices, systems, and methods in various aspects may notify or alert a user of the surgical device when a structural artifact of concern is detected within the surgical field during a surgical procedure. If a structural artifact of concern above a predetermined threshold for a particular surgical tool is detected, this detection may trigger a notification to the surgeon and/or activate a tool locking element to deactivate the surgical tool.

For example, the instrumented surgical device may integrate a sensor capable of blood flow detection within a surgical field with an energy application tool capable of dissecting or ligating tissue. In one non-limiting aspect, if the surgical tool is situated adjacent to a surgical field within which an unacceptably high blood flow is detected by the integrated sensor, the sensor may generate an alarm signal to the surgeon and/or deactivate the energy applicator. This alarm signal/deactivation may be sustained until the surgical tool is resituated adjacent to a surgical field with an acceptably low blood flow. In this example, the integrated blood flow sensor may reduce the risk of intraoperative bleeding during a surgical procedure,

In other aspects, a system is provided that includes the instrumented surgical device and associated modules to perform a surgical procedure. The system may include a sensor module to assess the readings from the integrated sensor of the instrumented surgical device to determine if a structural artifact is present within the surgical field, a control module to operate the surgical tool, and an alarm module to generate an alarm indication to the surgeon and/or deactivate the operation of the surgical tool when a structural artifact of concern is detected. The system may further include a GUI module to generate one or more forms used to receive inputs to the system and to deliver outputs from the system.

Detailed descriptions of various aspects of the instrumented surgical devices and non-medical instrumented devices, as well as associated systems and methods of using the devices are provided herein below.

### I. Overview of Surgical System

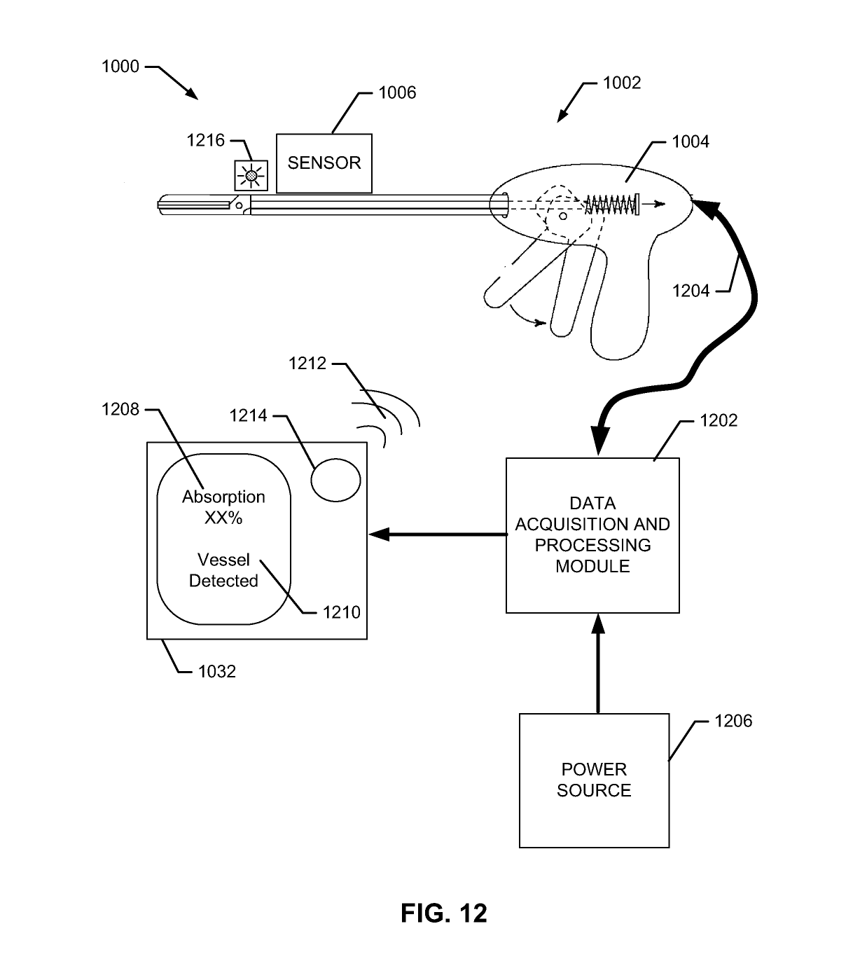

FIG. 12 is a schematic diagram representing an arrangement of the functional elements of a surgical system 1000 in one aspect. Referring to FIG. 12, the surgical system 1000 includes an instrumented surgical device 1002 used to perform a surgical procedure within a surgical field. The instrumented surgical device 1002 may include a surgical tool 1004 including, but not limited to, a grasper. The instrumented surgical device 1002 may also include a sensor 1006 including, but not limited to, a transmission light sensor such as a pulse oximeter. The sensor 1006 is operatively connected to the surgical tool 1004.

In one aspect, the sensor 1006 may be a separate device situated in proximity to the surgical tool 1004 in the surgical field during the surgical procedure. In another aspect, the sensor 1006 may be configured to reversibly attach to the surgical tool 1004. In yet another aspect, the sensor 1006 may be integrated into the structural elements of the surgical tool 1004. The sensor 1006 may be used to detect structural artifacts within a tissue in the surgical field including, but not limited to, blood vessels.

The surgical system 1000 may further include a data acquisition and processing module 1202 connected to the instrumented surgical device 1002 by a power cord 1204. The data acquisition and processing module 1202 may produce control signals used to operate the surgical tool 1004 and/or the sensor 1006. For example, the data acquisition and processing module 1202 may produce signals used activate a light source in the sensor 1006 used to detect the structural artifacts in the tissue. The data acquisition and processing module 1202 may also supply power obtained from the power source 1206 to the surgical tool 1004 and/or sensor 1006 of the instrumented surgical device 1002. In addition, the data acquisition and processing module 1202 may receive data signals from the sensor 1006 via the power cord 1204.

The data acquisition and processing module 1202 may also process the data signals received from the sensor to determine a characteristic of the tissue. For example, the characteristic of the tissue may be the percent absorption of light of a predetermined wavelength by the tissue. This characteristic of the tissue may be communicated to a display 1032 viewable by the surgeon while performing the surgical procedure in an aspect. For example, the percent absorption of light may be displayed continuously as a sensor readout 1208 on the display 1032.

In various aspects, the data acquisition and processing module 1202 may further process the sensor data to monitor for a structural artifact. In one aspect, the data acquisition and processing module 1202 may compare the processed sensor data to a threshold condition and issue an alarm signal if the processed sensor data exceeds the threshold condition. For example, the data acquisition and processing module 1202 may compare the percent absorption measured by the sensor 1006 to a stored value for a threshold condition corresponding to the minimum absorption associated with a structural artifact such as a blood vessel. In this example, if the measured percent absorption exceeds the threshold absorption, the module 1202 may issue an alarm signal to the display 1032.

Upon receiving the alarm signal, the display 1032 may communicate the alarm condition to the surgeon in any one or more of at least several ways. In one aspect, the sensor reading 1208 may be modified by changing the color of the displayed sensor reading 1208 or causing the sensor reading 1208 to flash, enlarge, or otherwise change appearance. In another aspect, a visual alarm display 1210 may be added to the display 1032. In yet another aspect, the display 1032 may further produce an auditory alarm 1212 such as an alarm tone using a speaker 1214.

Optionally, the system 1000 may further include an LED 1216 or other miniature indicator situated within the surgical field during the surgical procedure. The LED 1216 may illuminate, flash at one or more rates, change color, and/or generate any other visual indication to communicate the sensor reading and/or an alarm signal.

It is to be understood that FIG. 12 illustrates one non-limiting arrangement of elements of the surgical system 1000. Other arrangements and combinations of elements are possible in other aspects. For example, at least some of the functions of the data acquisition and processing module 1202 may be performed using a microprocessor or other processing device located with the sensor 1006 on the surgical instrument 1004. The functions of the display 1032 and the data acquisition and processing module 1202 may be implemented on a single device such as a personal computer. Other arrangements and positioning of devices and functions are possible in additional embodiments.

### II. Instrumented Surgical Device

In various aspects, the instrumented surgical device may include a surgical tool operatively connected to a sensor. “Operatively connected”, as used herein, refers to an arrangement of the surgical tool and the sensor to permit the concurrent operation of both the surgical tool and the sensor within the surgical field during a surgical procedure. In an aspect, the surgical tool and sensor may be situated in close proximity within the surgical field in order to effectuate the concurrent operation of the surgical tool and sensor.

In one aspect, the sensor may be a physically separate device from the surgical tool. In this aspect, the sensor may be situated within the surgical field and may operate concurrently with the surgical tool during the surgical procedure. In another aspect, the sensor may be detachably fastened to the surgical tool and operated concurrently with the surgical tool during the surgical procedure. In yet another aspect, the sensor may be integrated into one or more elements of the surgical tool and operated concurrently with the surgical tool.

The instrumented surgical device may further include a controller to control the use of the surgical tool by the surgeon and an optional display to communicate the output of the sensor to the surgeon in various other aspects. The surgical tool may include a functional element including, but not limited to, a pair of opposed jaws or blades, a laser, one or more electrodes, or other functional element to implement the surgical procedure. The sensor may be any known device appropriate for structural detection including, but not limited to, a blood flow detector, a tissue type detector, a material type detector, or any other detector capable of monitoring a surgical field and detecting a structural artifact of concern.

The surgical device may be used to cut, dissect, suture, seal, ligate, hook, grasp, apply a surgical appliance such as a clip, or perform any other function associated with a surgical procedure within a surgical field typically performed by a surgical tool. As the surgical tool is situated within a particular region of the surgical field and used to perform a surgical procedure, the sensor may monitor the surgical field to detect the presence of a structural artifact of concern. If a structural artifact is detected by the sensor, an alarm signal is produced by the sensor that may result in an alarm indication communicated to the surgeon to indicate an unsafe condition and/or the deactivation of the surgical tool.

In an aspect, the instrumented surgical device may be compatible for use in any surgical system or environment including, but not limited to, open surgery, endoscopic surgery including laparoscopic surgery and thoracoscopic surgery, angioplasty, stereotactic surgery, and robotic surgery.

A. Surgical Tool

In various aspects, the instrumented surgical device includes a surgical tool to perform a surgical procedure within a surgical field. Typically, the surgical tool may perform a function associated with a surgical procedure including, but not limited to, grasping, cutting, ligating, sealing, and any other function described herein above. The inclusion of the sensor with the instrumented surgical device provides the capability to assess the tissues within the surgical field to identify structural artifacts of concern such as blood flow exceeding a predetermined threshold level that may be impacted by the operation of the surgical tool. By assessing the sensor readings in real time as the surgical tool is situated within the surgical field and during the operation of the surgical tool, adverse events such as intraoperative bleeding and/or damage to sensitive tissues including, but not limited to, nervous tissues and urinary tract tissues may be reduced.

Any known surgical tool may be included in the instrumented surgical device without limitation. In an aspect, the surgical tool may be chosen from one or more of: a grasper, a dissector, a forceps, a clamp, a tissue sealing tool, a clip applier, a needle driver, a bone punch, a curette, a trocar, a biopsy punch, a scissors, a scalpel, an enucleator, a laser scalpel, a laser cauterization tool, an ultrasonic coagulation device, an ultrasonic ablation tool, an electrosurgical device, a laparoscopic probe, a surgical stapling device, a surgical sewing device, a biofragmentable anastomosis ring, a robotic surgical device, and any other suitable surgical tool.

The surgical tool may include a functional element to perform the function of the surgical tool and a controller to activate, deactivate, and otherwise modulate the operation of the surgical tool in response to inputs from the surgeon. In various aspects, the controller may modulate the operation of the surgical tool by any known means including, but not limited to: direct mechanical linkages such as hinged handles, pulleys, and push-rods; hydraulic actuators; electrical signals sent to electrical motors, actuators, or other electrical control devices. In an aspect, the controller may further be operatively connected to the sensor to modulate the operation of the surgical tool in response to the detection of a structural artifact of concern within the surgical field by the sensor.

In an aspect, the controller may be a hand-held controller including, but not limited to: a squeeze trigger, a handle, a lever, a button, and any other hand-held controller typically used in surgical tools, devices and/or systems. For example, the controller may be a pair of handles that may be grasped with varying degrees of pressure by the surgeon. In this example, the controller may respond to the pressure exerted by the surgeon by modulating a pressure exerted by the functional element of the device on a tissue within the surgical field. In addition, an integrated blood flow sensor may deactivate the controller if a blood flow in excess of a predetermined threshold is detected in the surgical field.

The functional elements and controllers in various aspects are described in detail herein below.

B. Functional Element

The functional element on the surgical tool may be used to perform the functions of the surgical tool including, but not limited to, cutting, sealing, dissecting, grasping, and hooking in various aspects. Non-limiting examples of functional elements within the surgical tool include one or more blades, clamps, hooks, jaws, energy applicators, or any other element or elements capable of implementing one or more functions of the surgical tool. Non-limiting examples of specific functional elements include a surgical scissors, a surgical hook, a blade or scalpel, a stationary cutting edge, a pair of scissor blades, a laser, an energy applicator, one or more electrodes, an electrical arc, suturing elements, a cauterizer or resistive heater, an ultrasonic transmitter, a pair of jaws, a rotating cutting edge, a reciprocating cutting edge, a water jet, a file, a scraper, any other functional element, and any combination thereof that may be incorporated into a surgical tool.

In an aspect, the type of functional element may influence the choice of sensor operatively connected to surgical tool. For example, functional elements that include at least two spatially separated parts including, but not limited to, a pair of jaws, a pair of scissor blades, or a pair of electrodes may be compatible with a transmission sensor that requires a signal transmitter and signal receiver situated on opposite sides of a tissue. In another example, a functional element that includes a single part including, but not limited to, a single blade or hook may be compatible with a reflection sensor that makes use of a signal transmitter and signal receiver situated on the same side of a tissue.

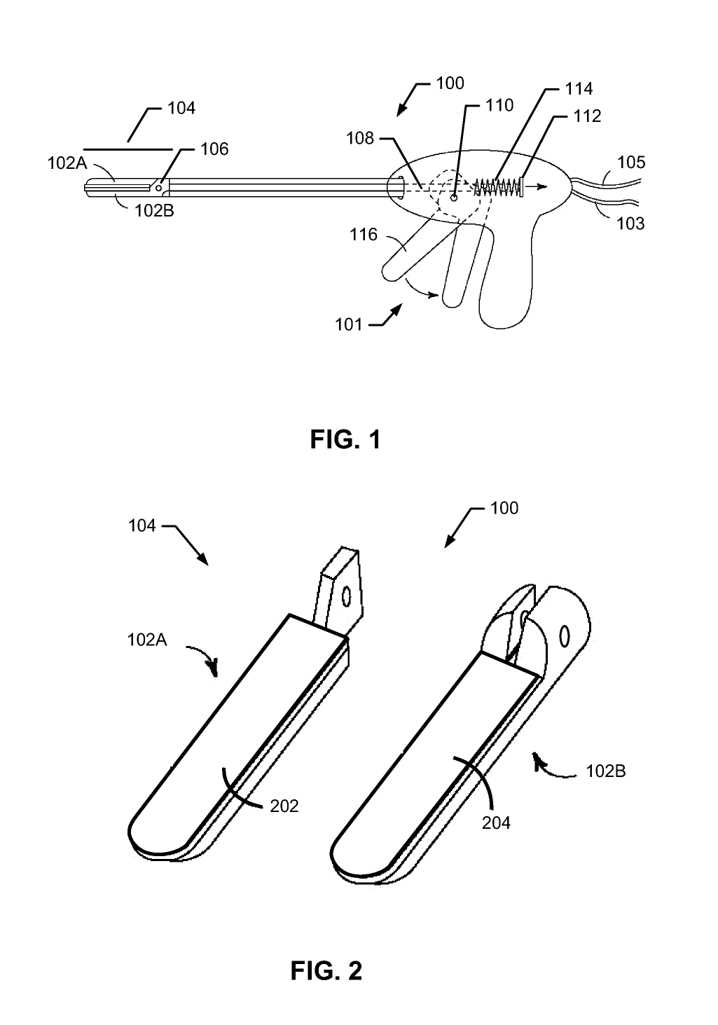

In one aspect, the surgical tool may include a first grasping jaw and a second grasping jaw. The grasping jaws may be oppositely situated to allow tissue or any other material to be grasped or held between the first and second grasping jaws. FIG. 1 is a side view showing an instrumented surgical device 100 that includes a functional element 104 made up of a first grasping jaw 102A and a second grasping jaw 102B, as well as a hand-held controller 101 operatively connected to the functional element 104.

Referring to FIG. 1, the first and second grasping jaws 102A/102B may be hinged together at a pin joint 106. The controller 101 may include a lever 116 attached to an actuator rod 108 at one end and constrained to rotate about a second pin joint 110. The free end 112 of the actuator rod 108 may be fitted with a biasing spring 114 such that the first and second grasping jaws 102A/102B are held closed when no force is applied to the controller 101 by the surgeon.

In an aspect, the controller 101 may be sensitive to pressure applied by the surgeon, such that the pressure applied to the controller 101 may cause the grasping jaws 102A/102B to apply a proportional amount of jaw pressure in order to grasp tissue (not shown) without incurring damage to the tissue. For example, a low amount of pressure applied to the controller 101 by the surgeon may result in low pressure clamping by the jaws 102A/102B. Alternatively, a high amount of pressure applied to controller 101 by the surgeon may result in high pressure clamping by the grasping jaws 102A/102B. The grasping jaws 102A/102B may grasp and manipulate the tissue to allow positioning without excessive tissue damage and/or to perform high pressure clamping for tissue sealing.

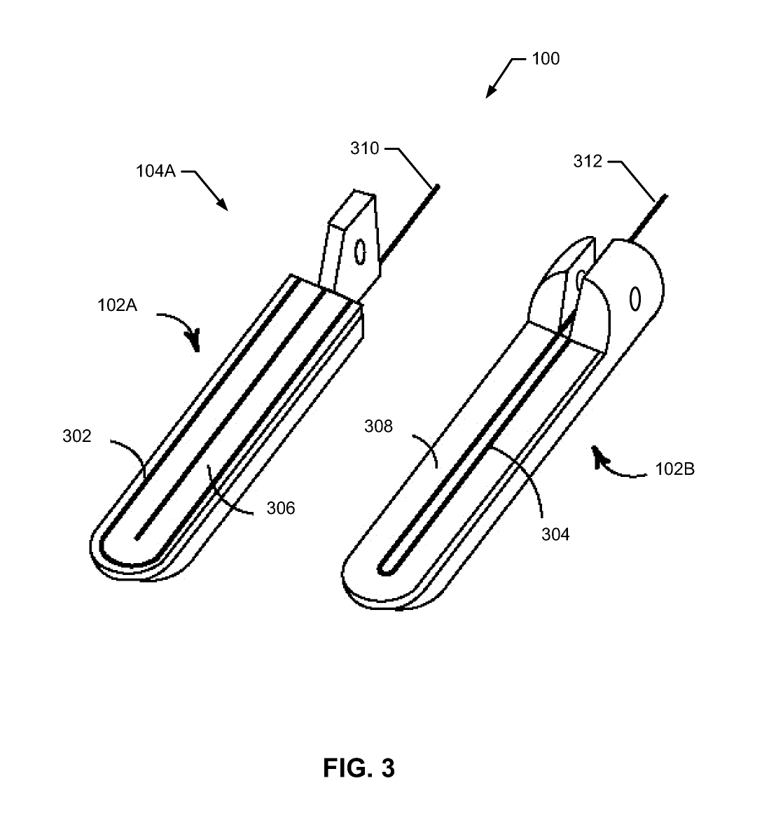

The first and second grasping jaws 102A/102B may be any known shape and size without limitation. In one aspect, the first and second grasping jaws 102A/102B may include jaw surfaces 202 and 204 in the form of elongate flat plates with rounded tips, as illustrated in FIG. 2. In other aspects (not shown), the first and second grasping jaws 102A/102B may have pointed, rounded, or other tip shapes. In other additional aspects (not shown), the first and second grasping jaws 102A/102B may be uniformly broad, uniformly narrow, may taper to a smaller width at the tip, may expand to a broader tip, and any other jaw shape. The jaw surfaces 202 and 204 may be planar as illustrated in FIG. 2 or may be concave, convex, ridged, hollow, or may have any other shape known in the art. The jaw surfaces 202 and 204 may incorporate surface texturing including, but not limited to, a plurality of raised points, bumps, ridges, or any other known surface texture. The jaw surfaces 202 and 204 may further incorporate textured edges situated around their lateral perimeters in the form of serrated edges, toothed edges, or any other known edge texture.

In another aspect, the instrumented surgical device 100 may be an energy applicator and/or an energy applicator incorporated into another surgical tool. The energy applicator may be any known energy applicator including, but not limited to, a laser, an ultrasound transmitter, a plasma source, a cryogenic source, one or more electrodes, and any other known energy applicator. The energy applicator may transfer energy to or from a tissue within the surgical field in order to implement a function such as cauterization, tissue sealing, tissue ablation, tissue stimulation, and any other known function of known energy application devices.

In one aspect, the energy applicator may be a laser. The laser energy may be produced by an external source and transferred to the surgical field using an optical element including, but not limited to, an optic fiber. In other aspects, the laser energy may be produced by an internal source including, but not limited to, an LED laser that is situated in close proximity to the surgical field. The wavelength, fluence, power, and any other known relevant laser parameter may be selected according to the desired function of the laser and according to known practices in the art. A laser energy applicator may be used to implement a variety of surgical tool functions including, but not limited to, a laser scalpel, a laser ablation tool, a photothermal ablation tool, a photoacoustic ablation tool, and any other known function of a laser energy applicator.

In another aspect, the energy applicator may be one or more electrodes. The electrodes may be provided in a variety of other forms without limitation. In one aspect, a single electrode may be provided as a functional element, or a single electrode may be incorporated into the functional element of another surgical tool to implement a monopolar surgical tool function. This single electrode may be incorporated into any surgical tool without limitation. In another aspect, two electrodes may be provided as a functional element, or two electrodes may be incorporated into the functional element of another surgical tool to implement a bipolar surgical tool function.

In one aspect, if the energy applicator is one or more electrodes, the one or more electrodes may transfer electrical energy in the form of electrical charge, electrical voltage, electrical current, and/or any other known electrical quantity into a tissue within the surgical field. Electrical energy may be supplied by an external electrical source including, but not limited to, an external power source or an internal power source. The external power source may be any known external power source including, but not limited to: a battery, an AC power source, a DC power source, a current source, a voltage source, and any other known external power source. The internal power source may be any known internal power source including, but not limited to, a battery, an inductive power source, a capacitor, and any other known internal power source. The electrical energy conducted through the tissue may supply the energy used to stimulate, ablate, or seal the tissue in various aspects.

For example, the first and second grasping jaws 102A/102B illustrated in FIGS. 1 and 2 may further include a first electrode 202 and a second electrode 204, respectively, as illustrated in FIG. 3 in a disassembled view. The resulting a functional element 104A, a bipolar grasper, may deliver electrical current through a tissue situated between the first and second grasping jaws 102A/102B. The electrodes 302 and 304 may be electrically connected to a first conductive plate 306 and a second conductive plate 308, respectively, as illustrated in FIG. 3. The conductive plates 306/308 may contact a region of tissue situated between the grasping jaws 102A/102B, and deliver an electrical current when connected to a power source (not shown) via conductive leads 310 and 312.

In an aspect, the electrodes 302 and 304 may be shaped as inner or outer u-shaped rings, as illustrated in FIG. 3, or may be shaped as linear strips, plates, screens, or any shape or conformation on the grasping jaws 102A and 102B. The electrodes 302 and 304 may be configured such that they may both contact the same tissue on opposite sides.

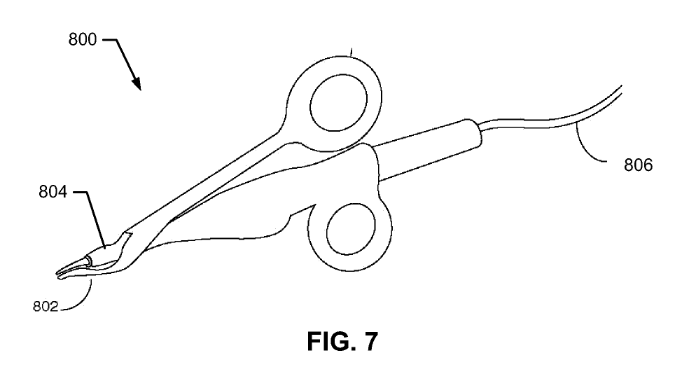

In another aspect, the surgical tool may include a surgical scissors tool 800, as seen in FIG. 7. The surgical scissors tool 800 may be used to grasp or clamp a tissue or blood vessel. In one aspect, the surgical scissors tool 800 may have an ultrasound Doppler probe 804 imbedded within the scissors 802.

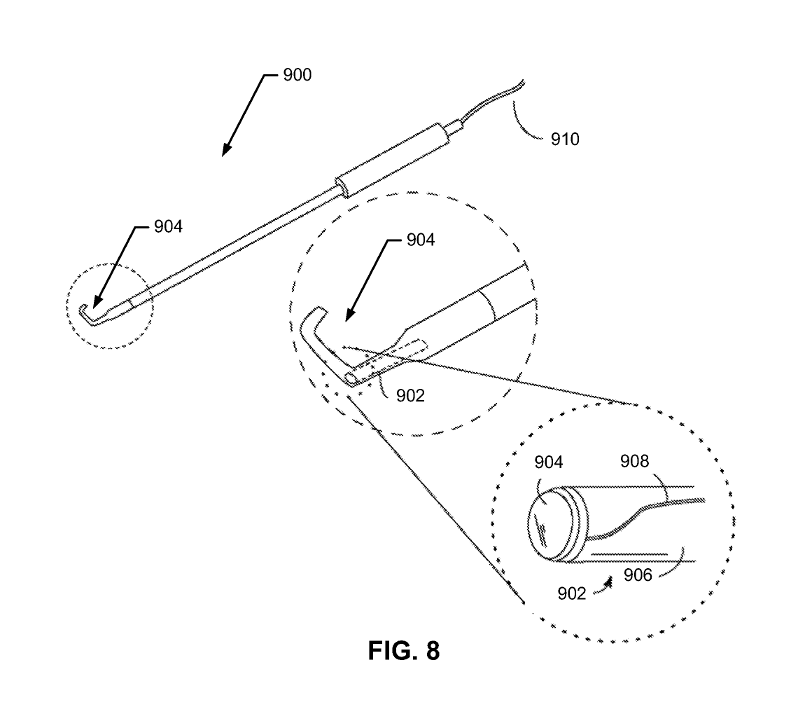

In yet another aspect, the surgical tool may include a surgical hook tool 900, as illustrated in FIG. 8. In this aspect, the hook 904 may be used to hook over a tissue or blood vessel to determine the structural artifact. In one aspect, the surgical hook 904 may be used to sense blood flow within the tissue or vessel. Referring back to FIG. 8, the surgical hook 904 may include an ultrasound Doppler probe 902.

C. Sensor

In various aspects, the instrumented surgical device may include a sensor operatively connected to the surgical tool. The sensor may monitor the surgical field for a structural artifact as described herein previously. Non-limiting examples of sensors suitable for use in the instrumented surgical device include an optical sensor, an infrared detector and receiver, a pulse oximeter, an ultrasound probe, an ultrasound Doppler probe, an acoustic Doppler velocimeter, a laser Doppler velocimeter, a photoacoustic sensor, a magnetic flow meter, a thermographic sensor, radar, a sonographic sensor, a magnetometer, or any other sensor that may be used to detect a surgical artifact.

The sensor may detect a variety of structural artifacts within the surgical field without limitation. In one aspect, the structural artifact may be directly detected using variety of sensors including, but not limited to, a blood flow detector, a tissue type detector, a material type detector, and any other detector capable of monitoring or detecting a structural artifact within the surgical field. In another aspect, the structural artifact that may be indirectly detected using measurements of other structural artifacts including, but not limited to, blood flow, blood or tissue oxygenation, or any other relevant structural artifact.

For example, a sensor may be used to detect one or more properties of a blood vessel. In this aspect, a blood flow detector may monitor or detect any one or more properties of the blood vessel including, but not limited to: a presence of a blood vessel, a size of the vessel, a speed of the blood flow through the vessel, a vessel orientation, and a vessel O2 saturation blood flow within the surgical field. In an aspect, the flow speed of the vessel may be used to estimate the size of the vessel. In one aspect, a blood vessel larger than a threshold vessel diameter within the surgical field may be detected and may further trigger an alarm signal to the surgeon.

In another aspect, the sensor may be used to detect the type of tissue or organ in the surgical field. Tissue, organ, or system types that may be detected include, but are not limited to, bone, fat, muscle, tendon, ligament, epithelial, dermis, epidermis, vascular, neural, cancerous tissue, liver, respiratory tract (lung, trachea), gastrointestinal (stomach, intestine), urinary tract (ureters, bladder, kidney), any other type of tissue or organ within the surgical field, or any combination. For example, a sensor operatively connected to an ablation device may be used to detect cancer tissue within the surgical field. In this aspect, if the sensor fails to detect cancer tissue within the surgical field, an alarm signal may be triggered to prevent the surgeon from ablating healthy tissue.

When a structural artifact is detected by the sensor, the sensor may generate an alarm signal. In an aspect, a structural artifact may be detected if a specific threshold is reached. For example, if the sensor detects blood flow in a blood vessel within the surgical field, the sensor may generate an alarm signal when the blood flow is above a set threshold indicating that the blood vessel may be too large for the specific surgical tool. In another aspect, an alarm signal may be generated in the absence of a structural artifact within the surgical field.

In various aspects, the sensor may be a transmission sensor, defined herein as a sensor that includes a sensing signal source and a sensing signal receiver situated on opposite sides of a tissue within a surgical field. Because the transmission sensor requires that the sensing signal source and sensing signal receiver be situated on opposite sides of the tissue, the transmission sensor may be suitable for use with surgical tools that include at least two spatially separated parts in the functional element of the surgical tool. Non-limiting examples of surgical tools suitable for integration with a transmission sensor include: a grasper, a forceps, a clamp, a tissue sealing tool, a clip applier, a needle driver, a bone punch, a biopsy punch, a scissors, and a bipolar forceps.

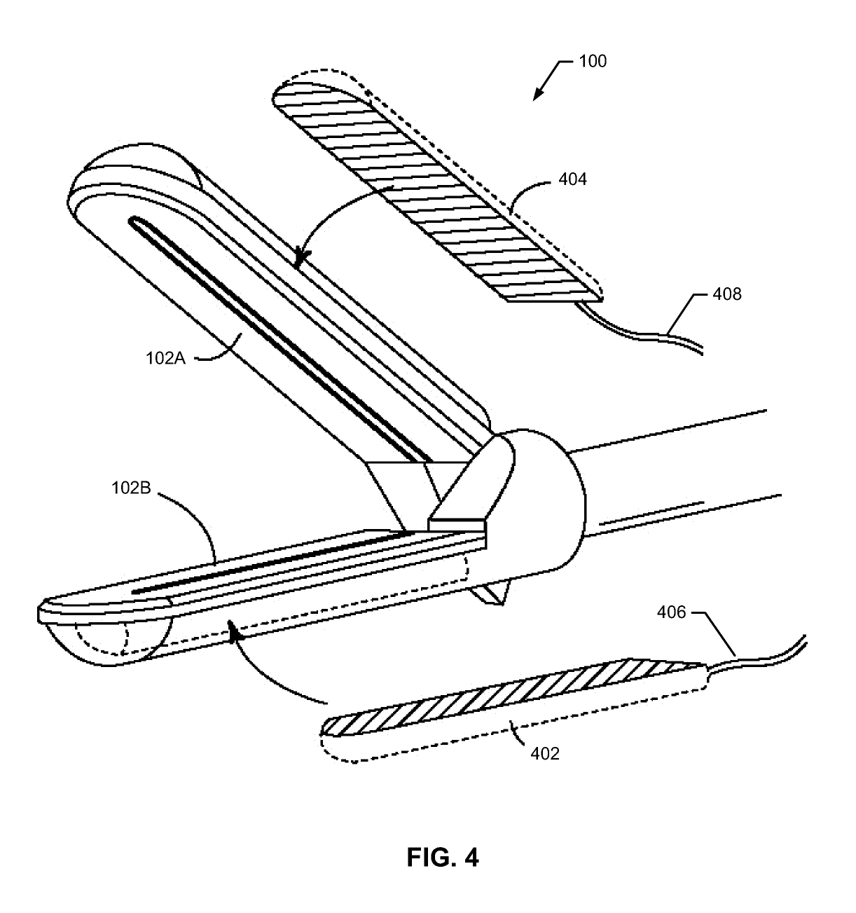

In one aspect, the structural detection element may be an optical sensor. FIG. 4 is an illustration of an optical sensor integrated into a functional element in the form of first and second grasping jaws 102A and 102B as previously discussed herein above and illustrated in FIG. 1. As illustrated in FIG. 4, the optical sensor may include an optical transmitter 402 integrated into the second grasping jaw 102B and an optical receiver 404 integrated into the first grasping jaw 102A. In other aspects the location of the optical transmitter 402 and optical receiver 404 may be reversed, or these elements may be situated elsewhere on the surgical tool 100.

Referring again to FIG. 4, the optical transmitter 402 may be connected to an external light source (not shown) via an efferent optical cable 406 operatively connected to the light source and optical transmitter 402 at opposite ends. Similarly, light received by the optical receiver 404 may be carried out of the surgical field to a data processing element (not shown) via an afferent optical cable 408 operatively connected to data processing element and optical receiver 204 at opposite ends. In an additional aspect (not shown), the signal beam 504 and the response beam 506 may be transferred from an external light source and to an external light sensing device, respectively, via a single optical cable.

The characteristics of the light produced by the light source and used by the optical sensor may be selected based on known properties of the light in the contexts of sensing a desired structural artifact. “Light”, as used herein, refers to any electromagnetic radiation with a wavelength and/or frequency falling within any light spectrum including, but not limited to: the visible light spectrum (wavelength=380 nm to 700 nm), the infrared (IR) light spectrum (wavelength=740 nm to 3×10⁵ nm), the near-infrared (NIR) light spectrum (wavelength=750 nm to 1400 nm), and the ultraviolet light spectrum (wavelength=10 nm to 380 nm). For example, any known wavelength suitable for sensing the desired structural artifact may be used by the optical sensor including, but not limited to, ultraviolet light, near-ultraviolet light, visible light, near-infrared light, and infrared light. In various aspects, the wavelength of light produced by the light source may be selected based on any one or more of at least several factors including, but not limited to: high transmissivity through many biological tissues; differential or specific absorption by a tissue, cell, or molecule associated with a tissue and/or cell such as hemoglobin; differential or specific absorption of a particular condition of a tissue, cell, or molecule associated with a tissue and/or cell such as oxygenated hemoglobin and deoxygenated hemoglobin.

In one aspect, the light produced by the light source may have a wavelength ranging between about 600 nm and about 1400 nm. In other aspects, the light produced by the light source may have a wavelength ranging between about 600 nm and about 700 nm, about 650 nm and about 750 nm, about 700 nm and about 800 nm, about 750 nm and about 850 nm, about 800 nm and about 900 nm, about 850 nm and about 950 nm, about 900 nm and about 1000 nm, about 950 nm and about 1050 nm, about 1000 nm and about 1100 nm, about 1050 nm and about 1150 nm, about 1100 nm and about 1200 nm, about 1150 nm and about 1250 nm, about 1200 nm and about 1300 nm, about 1250 nm and about 1350 nm, and about 1300 nm and about 1400 nm.

In various aspects, a wavelength that is highly absorbed by oxygenated and/or deoxygenated hemoglobin may be produced by the light source including, but not limited to, wavelengths from the red spectrum (620 nm-750 nm) and the near-infrared spectrum (750 nm-1400 nm). In one aspect, a wavelength of about 850 nm may be produced. In another aspect, a wavelength of about 660 nm may be produced. In yet another aspect, a wavelength of about 895 nm, about 905 nm, about 910 nm, or about 940 nm may be produced. Without being limited to any particular theory, the absorption of red and near-infrared wavelengths by hemoglobin is known to vary as a function of the percent oxygenation of the hemoglobin.

In one additional aspect, the light source may produce light at a single wavelength. In another additional aspect, the light source may produce light at two or more wavelengths. In this additional aspect, the two or more wavelengths may be produced simultaneously or alternatively may be produced separately and sequentially in a repeating pattern.

In another additional aspect, the light source may produce two wavelengths to implement a pulse oximetry method. Without being limited to any particular theory, the pulse oximetry method measures the absorption of light at a red wavelength of about 660 nm and at a near-infrared wavelength ranging from about 895 nm to about 940 nm. In this method, the calculated ratio of the absorption of the red wavelength and the near-infrared wavelength may be used to determine the oxygenation of the hemoglobin in the blood using a known correlation of this absorption ratio to blood oxygenation.

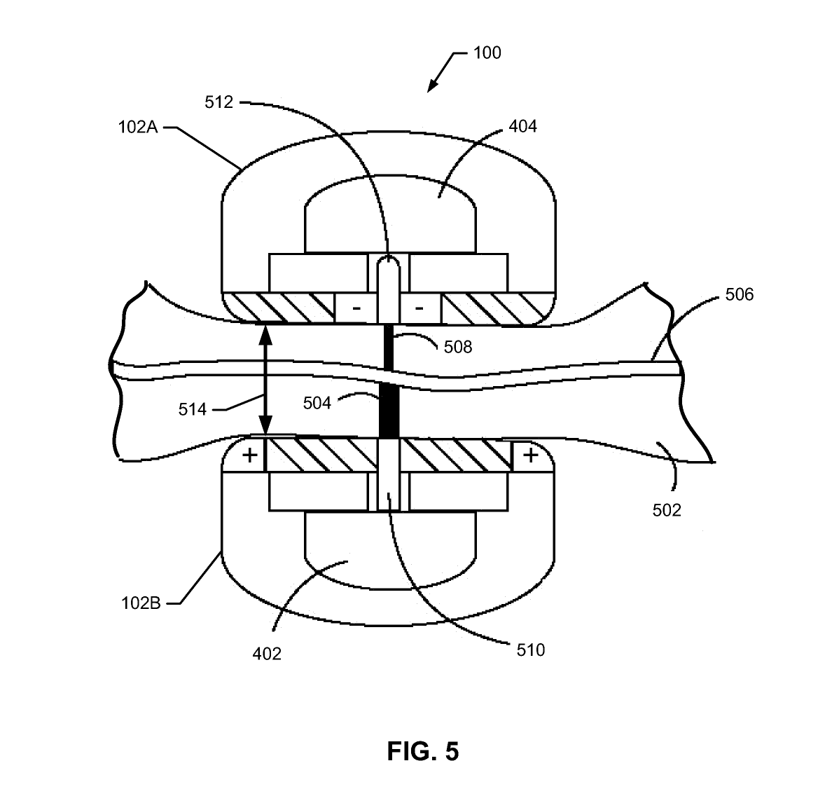

FIG. 5 is a cross-sectional view of the optical sensor illustrated in FIG. 4 with a tissue segment 502 situated between the first and second grasping jaws 102A and 102B. In this aspect, the optical transmitter 402 may transmit a signal beam 504 such as a near-infrared beam into the tissue segment 502. When the signal beam 504 passes through the tissue segment 502, at least one characteristic of the signal beam 504 including, but not limited to, light intensity may be altered by one or more aspects of the tissue segment 502 and/or structural artifacts situated within the tissue segment 502. For example, as illustrated in FIG. 5, if a blood vessel 506 is situated within the tissue segment 502, interference caused by the Doppler effect of blood cells passing through the vessel 506 may reduce the intensity of the signal beam 504 within the tissue segment 502. Due to this interference, the intensity of the response beam 508 emerging from the tissue segment opposite to the optical transmitter 402 may be reduced relative to the intensity of the signal beam 504.

Referring again to FIG. 5, the response beam 508 may be captured by the optical receiver 404 and transmitted to the data processing element (not shown) via an afferent optical cable 408 (not shown). Post-processing of the response beam 508 may be used to quantify one or more properties of the blood vessel 506 including, but not limited to, the presence of a vessel 506, the size of the vessel 506, the speed of the blood flow through the vessel 506, vessel orientation, vessel O₂ saturation and any other relevant property of the blood vessel 506. The one or more properties of the vessel 506 quantified by the optical sensor may be used to determine whether the surgeon may safely proceed with a function of the surgical tool 100 including, but not limited to, pressure clamping of the tissue 502 and/or tissue sealing using the surgical tool 100.

As illustrated in FIG. 5, the optical transmitter 402 may be situated directly across from the optical receiver 404 on the opposite side of the tissue 502 such that transmitted light may be detected. The signal beam 504 produced by the optical transmitter 200 may pass through a transmitter slit 510 formed through the material of the second grasping jaw 102B. The response beam 508 emerging from the tissue 502 may be captured by the optical receiver 404 through a receiver slit 512 formed within the material of the first grasping jaw 102A so that only light transmitted between grasping jaws 102A and 102B through the tissue 502 may be recorded.

The separation distance 514 between the optical transmitter 402 and the optical receiver 404 may range from about 0.1 mm to about 15 cm. In various aspects, the separation distance 514 between the optical transmitter 402 and the optical receiver 404 may range from about 0.1 mm to about 1 mm, from about 0.5 mm to about 5 mm, from about 2.5 mm to about 1 cm, from about 5 mm to about 2 cm, from about 1 cm to about 3 cm, from about 2 cm to about 4 cm, from about 3 cm to about 5 cm, from about 4 cm to about 6 cm, from about 5 cm to about 7 cm, from about 6 cm to about 8 cm, from about 7 cm to about 9 cm, from about 8 cm to about 10 cm, from about 9 cm to about 11 cm, and from about 10 cm to about 15 cm.

In one aspect, the signal beam 504 may be produced by an external light source (not shown) and the detected response beam 508 may be interpreted by any known light sensing device including, but not limited to, an external diode array spectrometer. In another aspect, the signal beam 504 may be produced by a local light source including, but not limited to, a near-infrared LED device situated within the optical transmitter 402; in this other aspect, the efferent optical cable 406 may be used to supply power to the local light source rather than to transmit light.

In yet another aspect, the response beam 508 may be interpreted by an external light sensing device including, but not limited to, an external diode array spectrometer situated outside of the surgical field. In another additional aspect, the response beam 508 may be interpreted by a light sensing device situated within the optical receiver 404 including, but not limited to, a diode array spectrometer. In this other additional aspect, the afferent optical cable 408 may be used to supply power to the light sensing device rather than to transmit light.

In one non-limiting example, the optical transmitter 402 may be an infrared LED producing light pulses with a wavelength of about 850 nm and the optical receiver 404 may be an IR photoreceptor. In another non-limiting example, the optical transmitter 402 may be a pair of LEDs including an IR LED producing light pulses at a wavelength of about 895 nm and a red LED producing light at a wavelength of about 660 nm; the optical receiver 404 may be a photodetector. In this example, the optical transmitter 402 may further include an LED drive to operate the pair of LEDs in an alternating pattern. The LED drive may further adjust the output of the pair of LEDs based on the output of the optical receiver 404 to enhance the resolution of the sensor output. In one aspect, the pair of LEDs may operate at a voltage ranging between about 3 V and about 5.5 V. In this example, the sensor output may be processed to obtain blood oxygenation using known optical oximetry methods.

In various other aspects, the optical sensor may be implemented in a reflection mode (not shown), rather than in the transmission mode illustrated in FIGS. 4 and 5. In the reflection mode, the optical transmitter 402 and the optical receiver 404 may be situated on the same side of the tissue 502. In this aspect, the response beam emerges from the same side of the tissue that previously received the signal beam; this response beam may include those portions of the signal beam that were reflected and/or scattered within the tissue 502. The properties of the reflected response beam may be influenced by structural artifacts situated within the tissue including, but not limited to, the presence of a vessel, the size of the vessel, the speed of the blood flow through the vessel, vessel orientation, vessel O₂ saturation.

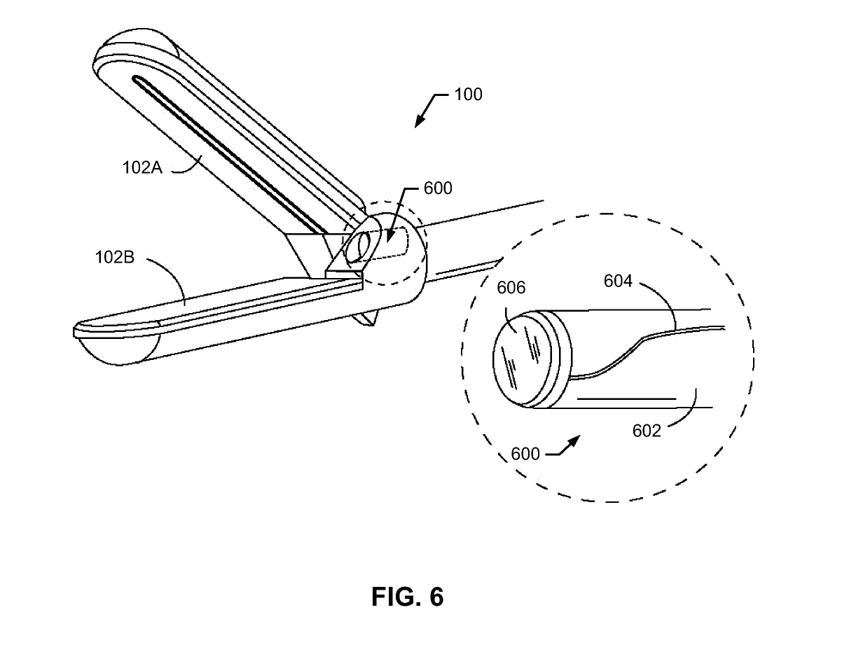

In additional to optical sensors, other sensor types may be integrated into the instrumented surgical device in various aspects without limitation. FIG. 6 is an illustration of an ultrasound Doppler probe 600 integrated into a functional element in the form of first and second grasping jaws 102A and 102B as previously discussed herein above and illustrated in FIG. 1. As illustrated in FIG. 6, the ultrasound Doppler probe 600 may include an ultrasound transceiver 606, a casing 602, and a signal wire 604 integrated at the base of the grasping jaws 102A/102B. Using known reflective Doppler methods, the probe 600 may analyze the region of tissue (not shown) resting against the probe 600. The ultrasound Doppler probe 600 may be connected to an external processing unit (not shown) that may evaluate structural artifacts including, but not limited to, blood flow and issue an alert signal if the blood flow or other structural artifact exceeds predetermined threshold values.

In one aspect, the ultrasound Doppler probe 600 may operate using ultrasound at a frequency ranging between about of about 5 MHz and 20 MHz. In various other aspects, the ultrasound Doppler probe 600 may operate using ultrasound at a frequency of about 5 MHz, about 8 MHz, about 10 MHz, and about 20 MHz. In an additional aspect, the ultrasound Doppler probe 600 may operate using ultrasound at a frequency of about 8 MHz. Typically, the ultrasound Doppler probe 600 may have a penetration distance of about 4 inches in depth or more, depending on the composition of the tissue.

In another aspect, a sensor 804 may be integrated into the functional element 802 of surgical scissors 800, as illustrated in FIG. 7. Power and signal emission and analysis may be enabled through the connection cable 806.

In another aspect, a Doppler probe 902 may be integrated within a surgical device 900 including a functional element in the form of a surgical hook 904 as illustrated in FIG. 8. Referring to FIG. 8, the ultrasound Doppler probe 902 includes an ultrasound transceiver 904, a casing 906, and an ultrasound signal wire 908 that may be integrated at the base of the surgical hook 904. Using reflective Doppler technology, the probe 902 may analyze the region of tissue (not shown) resting against the probe 902. The ultrasound Doppler probe 902 may be connected to an external processing unit (not shown) through an ultrasound connecting wire 910 that may evaluate blood flow and issue an alert signal if the blood flow or other structural artifact exceeds a predetermined threshold value for the device 900. In this aspect a surgeon may evaluate a surgical field targeted for dissection by placing the device 900 on the region prior to dissection by hooking.

E. Indicator

In various other aspects, the instrumented surgical device may further include an indicator operatively connected to the sensor. The indicator may be activated in response to an alarm signal generated by the sensor. Non-limiting examples of suitable indicators include a visual display, a speaker, a vibration generator, a tool locking element, or any other means of communicating the alarm signal to a user of the instrumented surgical device. In an aspect, the visual display may generate a visual alarm indication in response to the alarm signal. In another aspect, the speaker may generate an auditory alarm indication in response to the alarm signal. In another aspect, the vibration generator may generate a tactile alarm indication in response to the alarm signal. In yet another aspect, the tool locking element may be operatively connected to the surgical tool to deactivate the surgical tool; in this aspect, the tool locking element may be operatively connected to the controller.

In one aspect, the indicator may be situated on the instrumented surgical device within the surgical field. For example, the indicator may be a LED attached to the instrumented surgical device in proximity to the functional element 104. In this example, the LED indicator may illuminate, flash, change color, and/or provide another visual indication in response to an alarm signal. In another example, the LED indicator may provide a visual indication to communicate the sensor reading. In this other example, the LED indicator may display different colors, flash at different rates, and/or provide another visual indication to communicate the sensor reading. In an additional example, the LED indicator may flash at different rates as a function of the sensor reading and may additionally illuminate steadily in response to an alarm signal.

In another aspect, the indicator may be situated outside of the surgical field. Non-limiting examples of indicators situated outside of the surgical field include: a display on an external monitor screen, an external speaker that emits a tone in response to an alarm signal, and any combination thereof.

### III. Surgical System

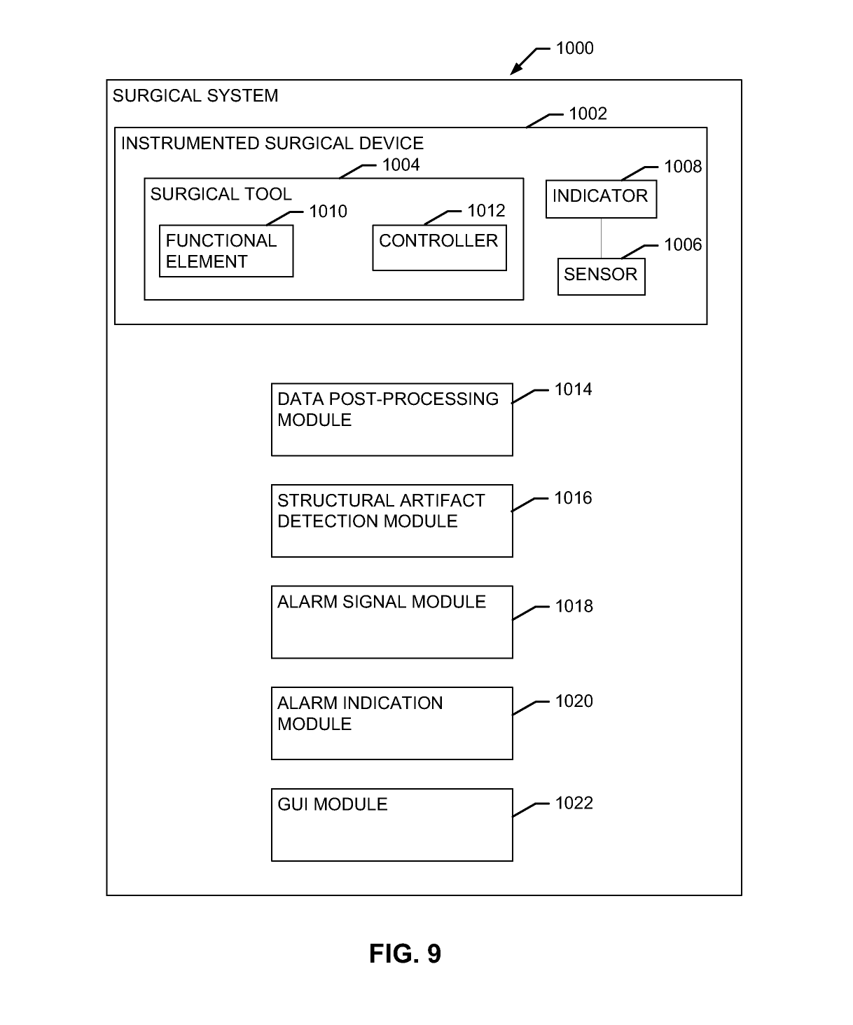

In various aspects, a surgical system is provided to perform a surgical procedure on a tissue within a surgical field of a patient. A block diagram representing the components of a surgical system 1000 is provided at FIG. 9. The surgical system 1000 includes an instrumented surgical device 1002 for implementing the surgical procedure and for concurrently monitoring the tissue within the surgical field to detect any structural artifacts during the surgical procedure. In an aspect, the instrumented surgical device 1002 is similar to the instrumented surgical devices described herein above.

The instrumented surgical device 1002 includes a surgical tool 1004 to implement the surgical procedure within the surgical field. The surgical tool 1004 includes a functional element 1010 to implement the surgical procedure and a controller 1012 to activate, deactivate and/or modulate the operation of the functional element 1010 of the surgical tool 1004. The functional element 1010 may include any of the functional elements described previously herein above including, but not limited to: one or more blades, clamps, hooks, jaws, energy applicators, and any combination thereof. The controller 1012 may include any one or more of the controllers described herein previously including, but not limited to: a squeeze trigger, a handle, a lever, a button, and any combination thereof. In one aspect, the controller 1012 may be a lever sensitive to forces and/or pressures applied by the surgeon during the performance of a surgical procedure as illustrated in FIG. 1 and described previously herein. In additional aspects, the controller 1012 may be modulated by other modules of the system 1000 including, but not limited to: a structural artifact module 1016, an alarm signal module 1018, an alarm indication module 1020, a GUI module 1022, and any combination thereof.

In an aspect, the surgical tool 1004 is operatively connected to a sensor 1006 to monitor the surgical field during the surgical procedure. Any one or more of the sensors described herein above may be suitable for use as the sensor 1006 in the system 1000 including, but not limited to: the optical sensor illustrated in FIGS. 4 and 5, the ultrasonic Doppler flow probe illustrated in FIG. 6, and any combination thereof. In various aspects, the sensor 1006 may be a transmission sensor such as an optical transmission sensor in which the transmitter and receiver of the sensor 1006 are situated on opposite sides of the tissue within the surgical field as illustrated in FIG. 5. In various other aspects, the sensor 1006 may be a reflective sensor such as an ultrasound Doppler flow probe in which the transmitter and the receiver of the sensor 1006 are situated on the same side of the tissue as illustrated in FIG. 6.

Referring again to FIG. 9, an optional indicator 1008 may be operatively connected to the sensor 1006 and/or other modules of the system 1000 to communicate any alarm indications resulting from the detection of a structural artifact in excess of a predetermined threshold condition as described previously herein. Any of the indicator devices described previously herein may be suitable for use in the system 1000. Non-limiting examples of suitable indicator devices include a visual indicator such as a light or other visual display; an auditory indicator such as a speaker to emit a tone; a vibratory indicator such as a shaker to vibrate at least a portion of the surgical tool 1004; a tool locking element operatively connected to the controller 1012 to deactivate the surgical tool 1004, and any combination thereof. In one aspect, the indicator 1008 may be situated within the surgical field with the functional element 1010 of the surgical tool 1004. In another aspect, the indicator 1008 may be situated with the display 1032 outside of the surgical field.

Referring again to FIG. 9, the system 1000 may further include a data post-processing module 1014 to process the raw sensor data received from the sensor 1006. The raw sensor data may typically include one or more voltage readings obtained from sensing elements including, but not limited to, one or more photodiode readouts, one or more ultrasonic sensor readouts, and any other known sensor readout.

In various aspects, the raw sensor data may be processed at a sample rate ranging between about 30 Hz and about 1000 Hz. The sample rate of the raw sensor data may influence the quality of the processed sensor data. For example, sensor data obtained at a relatively low sample rate may include more variations in the values due to the artifacts of various data processing methods that make use of local averaging or curve-fitting that are sensitive to the temporal resolution of the data; these artifacts may be particularly pronounced during movement of the surgical tool 1004 and/or sensor 1006. In one aspect, the raw sensor data may be processed at a sample rate of about 500 Hz.

The data post-processing module 1014 may perform any one of more of at least several known data processing methods to determine one or more characteristics of the tissue within the surgical field including, but not limited to: the presence of a blood vessel, the size of the vessel, the speed of the blood flow through the vessel, vessel orientation, and vessel O₂ saturation; and tissue types such as nervous tissues and urinary tract tissues. The data processing methods performed by the data post-processing module 1014 may depend upon any one or more of at least several factors including, but not limited to: the type of sensor 1006 incorporated into the system 1000, the type of surgical tool 1004 and/or surgical procedure to be performed by the surgical tool 1004; and the particular structural artifact to be detected during the monitoring of the surgical field during the surgical procedure. Non-limiting examples of data processing methods that may be performed by the data post-processing module 1014 include smoothing, averaging, normalizing, scaling, applying a calibration, unit conversion, arithmetical operations, analog-to-digital conversion, differentiation, integration, demuxing, image reconstruction, statistical analysis, frequency analysis such as fast Fourier transform and/or spectral analysis, and any other known data processing method.

In one non-limiting example, the data post-processing module 1014 may process the raw sensor data received from a pulse oximeter device. The pulse oximeter device may include an infrared (IR) LED that produces light at a wavelength of about 895 nm and a red LED that produces light at a wavelength of about 660 nm in an alternating pattern of flashes. The pulse oximeter device further includes one or more photodetectors to measure the intensity of the light transmitted through the tissue within the surgical field. The raw data received from the pulse oximeter device may include raw voltage readings from the one or more photodetectors corresponding to the intensity of the transmitted red light and the intensity of the transmitting IR light in a continuous train. The data post-processing module 1014 may separate the red light signals from the IR light signals in the raw signal data, convert these signals into percent absorption values, obtain the ratio of the percent absorption values, and convert the ratio into a percent oxygenation value for the blood flow detected by the sensor 1014.

The processed sensor data produced by the data post-processing module 1014 may be displayed using the display 1032. For example, if the sensor is a pulse oximeter, the percent oxygenation value may be displayed continuously on the monitor.

Referring again to FIG. 9, the system 1000 may further include a structural artifact detection module 1016 to analyze the processed data produced by the data post-processing module 1014 and identify any structural artifacts that may occur within the surgical field. The structural artifact may be detected using any known method associated with the sensor 1006 of the system 1000. For example, if the sensor 1006 is an optical transmission sensor, the structural artifact may be a blood flow rate characterized by a heightened reduction in the intensity of a signal light beam after passing though the tissue within the surgical field. Any data characterizing one or more detected structural artifacts within the surgical field are transferred to an alarm signal module 1018 for additional analysis.

The alarm signal module 1018 assesses the data received from the structural artifact detection module 1016 to determine whether the detected structural artifact(s) increase the risk of an adverse event including, but not limited to, intraoperative bleeding and/or damage to sensitive tissues such as nervous tissues. Although the structural artifact detection module 1016 may detect one or more structural artifacts, the characteristics of the detected structural artifact(s) may not pose any risk of an adverse event during the implementation of a surgical procedure by the system 1000. For example, the structural artifact detection module 1016 may detect blood flow within the surgical field, but the blood flow may be sufficiently low that no risk of intraoperative bleeding is incurred by the use of the surgical tool 1004.

In an aspect, the alarm signal module 1018 compares the data characterizing one or more structural artifacts to one or more predefined threshold conditions and generates an alarm signal if the data exceed the one or more predefined threshold conditions. The threshold condition selected for use by the alarm signal module may depend upon the particular type of sensor 1006 or structural artifact to be detected. For example, if the structural artifact detection module identifies a blood flow within the surgical field, the alarm signal module may compare the flow velocity characterizing the blood flow to a predetermined threshold flow velocity and issue an alarm signal if the flow velocity exceeds the threshold flow velocity. Additional predetermined threshold conditions may include a maximum blood vessel size that is compatible with a surgical tool 1004, a maximum percentage of volume within the surgical field that is nervous tissue, a maximum electrical current fluctuation within the surgical field indicative of nervous tissue, and any other suitable threshold condition.

In one non-limiting example, the sensor 1006 may be an infrared LED producing light at a wavelength of 850 nm and an IR photoreceptor situated on opposite sides of a tissue in a surgical field. The sensor output data produced by this sensor may be a percent absorption value representing the amount of the 850 nm light absorbed by the tissue. This sensor 1006 may be calibrated to determine a tissue absorption value measured through tissue lacking in blood vessels as well as a vessel absorption value measured through a blood vessel within the tissue. The threshold condition in this example may be an absorption value corresponding to a value between the tissue absorption value and the vessel absorption value. In one aspect, the threshold value may be the absorption value that is halfway between the vessel absorption value and the tissue absorption value. In another aspect, the threshold value may be a percentage of the vessel absorption value including, but not limited to: about 50%, about 60%, about 70%, about 80%, and about 90% of the vessel absorption value.

In another non-limiting example, the structural artifact detection module 1016 may detect an effective diameter of a blood vessel. “Effective diameter”, as used herein, refers to the maximum cross-sectional dimension of the blood vessel, and is influenced by the orientation of the blood vessel with respect to the surgical tool 1004. For example, if a blood vessel with a diameter of 7 mm oriented perpendicular to the surgical tool 1004 is detected, the effective diameter would be about 7 mm. However, if the same blood vessel was oriented at a non-perpendicular angle to the surgical tool 1004, the effective diameter would be larger than 7 mm. If the surgical tool is a electrosurgical device, for example, if the detected effective diameter of a vessel is larger than the maximum operational dimension of the electrosurgical device, the electrosurgical device may be unable to completely seal the blood vessel. In this example, the threshold condition may be the maximum operational dimension capable of treatment by the surgical tool 1004.

Referring again to FIG. 9, the system may further include an alarm indication module 1020 to produce one or more alarm indications in response to one or more alarm signals received from the alarm signal module 1018. In one aspect, the alarm indication module 1018 may produce an alarm indication in response to each alarm signal received from the alarm signal module 1018. In another aspect, the alarm indication module 1018 may produce an alarm indication in response to a minimum rate of alarm signals (signals/sec) received from the alarm signal module 1018. In yet another aspect, the alarm indication module 1018 may produce an alarm indication in response to an initial alarm signal received from the alarm signal module 1018 and may further maintain the alarm indication so long as a subsequent alarm signal is received from the alarm signal module 1018 after a time period that is less than a predetermined threshold time period. In still yet another aspect, the intensity of the alarm signal may be modulated in proportion to any one or more characteristics of the alarm signals received from the alarm signal module 1018 including, but not limited to: the rate of alarm signals, the elapsed time from the initial alarm signal during an active alarm condition, and any combination thereof.

The one or more alarm indications produced by the alarm indication module may be used to produce visual indications, auditory indications, vibrational indications, and/or may further be used to activate a tool locking element as described previously herein.

In an additional aspect, the system 1000 may further include a GUI module 1022 to transmit/receive one or more forms to receive inputs from the surgeon and to transmit output from the system 1000. The surgeon may interact with one or more forms generated by the GUI module 1022 to enter data and/or to make menu selections used to implement the surgical procedure using the system 1000.

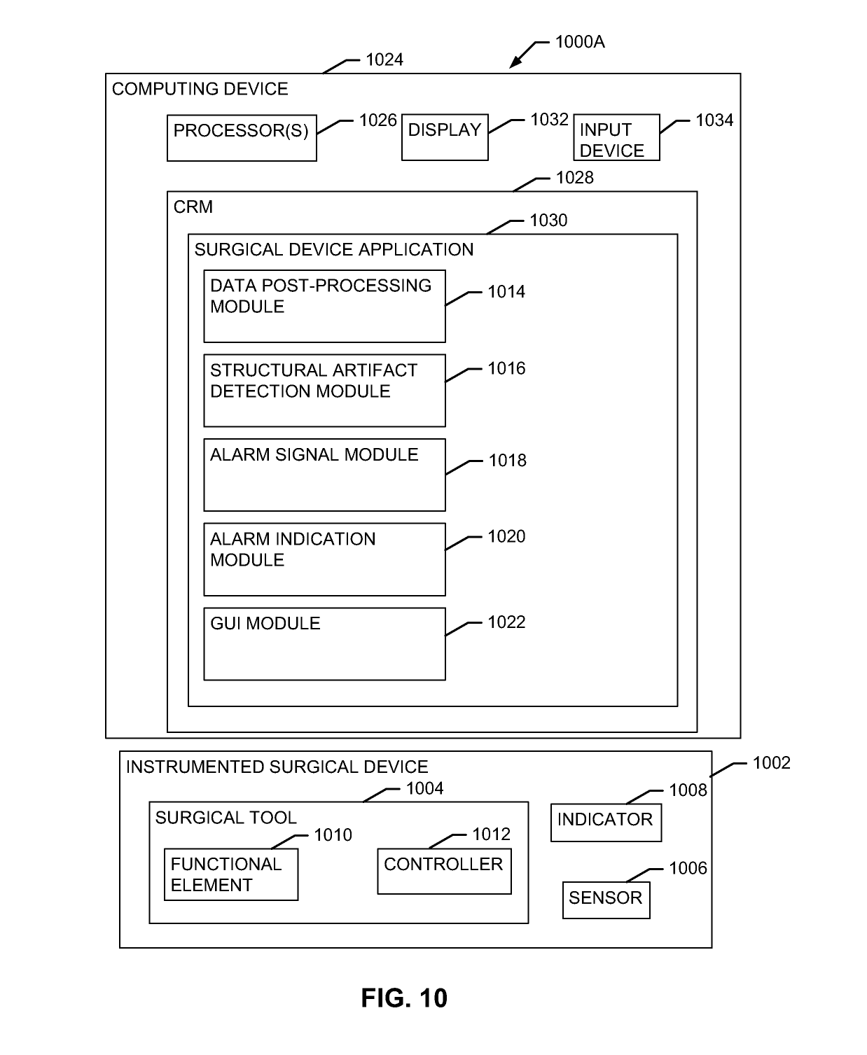

FIG. 10 is a block diagram illustrating a surgical system 1000A in another aspect. In this other aspect, the surgical system 1000A includes a computing device 1024 that includes one or more processors 1026 and a computer readable medium (“CRM”) 1028 configured with a surgical device application 1030. Non-limiting examples of a suitable computing device 1024 include a laptop computer, a personal digital assistant, a tablet computer, a standard personal computer, or any other known computing device. The computing device 1024 includes one or more processors 1026 and memory (not shown) configured to send, receive, and process data and/or communications from an operator of the system 1000A, such as a surgeon.

The CRM 1028 may include volatile media, nonvolatile media, removable media, non-removable media, and/or another available medium that can be accessed by the computing device 1024. By way of example and not limitation, computer readable medium 1028 comprises computer storage media and communication media. Computer storage media includes nontransient memory, volatile media, nonvolatile media, removable media, and/or non-removable media implemented in a method or technology for storage of information, such as computer readable instructions, data structures, program modules, or other data. Communication media may embody computer readable instructions, data structures, program modules, or other data and include an information delivery media or system.

The surgical device application 1030 includes instructions or modules that are executable by the one or more processors 1026 to enable the implementation of a surgical procedure using the instrumented surgical device 1002. The surgical device application 1030 stored on the CRM 1028 may include any one or more of the modules described herein previously including, but not limited to: the data post-processing module 1014, the structural artifact detection module 1016, the alarm signal module 1018, the alarm indication module 1020, and the GUI module 1022.

In various aspects, the CRM 1028, the surgical device application 1030, and/or the one or more processors 1026 may be situated within a computing device 1024 located outside of the surgical field. In various other aspects, the CRM 1028, the surgical device application 1030, and/or the one or more processors 1026 may be situated within a computing device 1024 situated within the instrumented surgical device 1002. In various additional applications, the CRM 1028, the surgical device application 1030, and/or the one or more processors 1026 may be situated within both a first computing device 1024 located outside of the surgical field and a second computing device 1024A situated within the instrumented surgical device 1002. For example the instrumented surgical device 1002 may include a microchip that includes one or more processors to execute at least a portion of the instructions of one or more of the modules of the surgical device application.

The computing device 1024 may further include a display 1032 configured to display data and/or one or more forms generated by the GUI module 1022. Non-limiting examples of devices suitable for use as a display 1032 include a computer monitor and a touch screen. The computing device 1024 may further include an input device 1034 including, but not limited to, a keyboard and/or a pointing device such as a mouse, a trackball, a pen, or a touch screen. The input device 1034 is configured to enter data into or interact with the forms generated by the GUI module 1022 used to implement the operation of the system 1000A. In an embodiment, the display 1032 and input device 1034 may be a single integrated device, such as a touch screen. The forms generated by the GUI module 1022 may enable the operator of the system 1000A to interact with menus and other data entry forms used to control the operation of the system 1000A.

### IV. Surgical Method

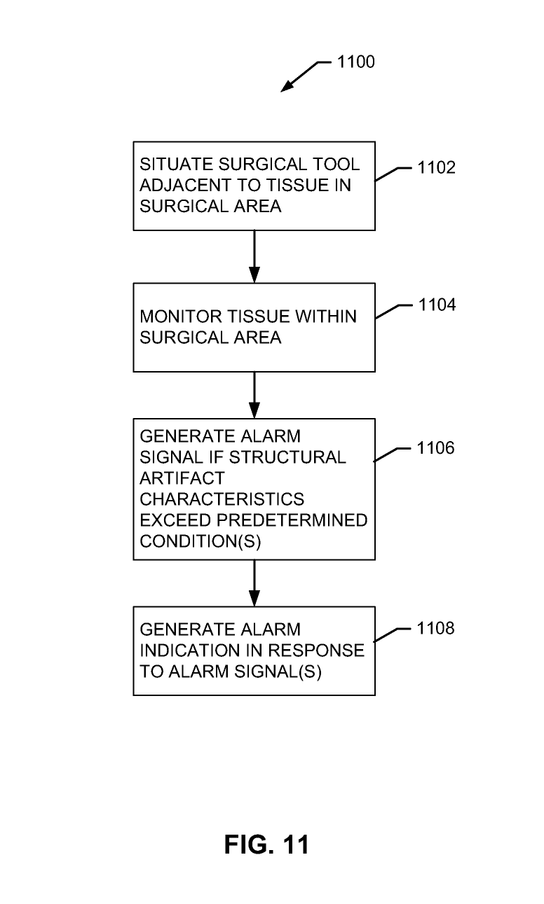

In an additional aspect, the instrumented surgical device and associated system may be used to implement a surgical method for performing a surgical procedure. A flowchart illustrating the steps of the method 1100 in one aspect is provided as FIG. 11. The method 1100 makes use of an instrumented surgical device similar to any of the devices described herein previously; the instrument surgical device includes a surgical tool with a controller operatively coupled to a functional element as well as a sensor operatively coupled to the surgical tool.

Referring back to FIG. 11, the method 1100 includes situating the instrumented surgical tool, in particular the functional element of the instrumented surgical tool, adjacent to the tissue within the surgical field at step 1102. The sensor of the instrumented surgical tool is used to monitor the tissue within the surgical field at step 1104. At step 1106, an alarm signal is generated by the instrumented surgical tool if a structural artifact such as a blood flow is detected by the sensor within the surgical field. At step 1106, the alarm signal may be generated if the sensor data characterizing the structural artifact exceeds one or more predetermined threshold conditions as described herein previously. In response to the alarm signal, an alarm indication may be generated at step 1108 to communicate that a structural artifact of concern was detected within the surgical field by the sensor. As described herein previously, the alarm indication may be a visual indication, an auditory indication, a vibratory indication, or the alarm indication may trigger the deactivation of the surgical tool in various aspects.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

1. An instrumented surgical device comprising:
a surgical tool to perform a surgical procedure within a surgical field; and
a sensor operatively connected to the surgical tool, wherein the sensor monitors the surgical field for a structural artifact.

2-37. (canceled)

38. A method of performing a surgical procedure on a tissue within a surgical field, comprising:
approaching the tissue with an instrumented surgical device comprising a sensor operatively attached to a surgical tool;
sensing a structural artifact within the tissue using the sensor;
sending an alarm signal from the sensor to an indicator if the structural artifact exceeds a predetermined threshold condition; and
generating an alarm indication in response to the alarm signal using the indicator.

39-51. (canceled)
